(19) United States
(12) Patent Application Publication
(10) Pub. No.: US 2026/0264035 A1
(43) Pub. Date: Sep. 10, 2026

(54) HYDROCARBON PRODUCTION APPARATUS

(71) Applicant: IHI Corporation, Tokyo (JP)

(72) Inventors: Kentaro NARIAI, Tokyo (JP); Kimihiro SAWA, Tokyo (JP); Atsushi NONOMURA, Tokyo (JP); Hiroyuki KAMATA, Tokyo (JP)

(73) Assignee: IHI Corporation, Tokyo (JP)

(21) Appl. No.: 19/661,840

(22) Filed: Apr. 29, 2026

Related U.S. Application Data
(63) Continuation of application No. PCT/JP2024/038631, filed on Oct. 30, 2024.

(30) Foreign Application Priority Data
Nov. 27, 2023 (JP) 2023-199655

(51) Int. Cl. B01J 8/02; C10G 2/00

(52) U.S. Cl. CPC B01J 8/0278; C10G 2/50; B01J 2208/00548; B01J 2208/00893

(57) ABSTRACT

Provided is a hydrocarbon production apparatus, including: a reaction device, which has an inlet, an outlet, and one or a plurality of supply ports formed between the inlet and the outlet, and in which a catalyst that promotes an exothermic reaction between hydrogen and carbon dioxide is accommodated; a first hydrogen supply unit that supplies the hydrogen into the reaction device through the inlet of the reaction device; and a carbon dioxide supply unit that supplies the carbon dioxide into the reaction device through the inlet and the one or the plurality of supply ports of the reaction device.

### CROSS REFERENCE TO RELATED APPLICATIONS

This application is a continuation application of International Application No. PCT/JP2024/038631, filed on Oct. 30, 2024, which claims priority to Japanese Patent Application No. 2023-199655, filed on Nov. 27, 2023, the entire contents of which are incorporated by reference herein.

### BACKGROUND ART

### Technical Field

The present disclosure relates to a hydrocarbon production apparatus. This application claims the benefit of priority to Japanese Patent Application No. 2023-199655 filed on Nov. 27, 2023, and contents thereof are incorporated in this application.

### Related Art

Fossil fuels such as coal, heavy oil, and extra heavy oil are combusted in plants such as a thermal power plant, a steel plant, and a boiler. Thus, an exhaust gas containing any one or both of carbon dioxide and carbon monoxide generated as a result of the combustion of a fossil fuel is exhausted from the plant to the air. Carbon dioxide and carbon monoxide are considered as causes of the global warming, and hence there has been developed a technology of collecting any one or both of carbon dioxide and carbon monoxide from the air.

Moreover, as a technology for effectively using the collected carbon dioxide, for example, in Patent Literature 1, there is disclosed a technology of producing methane by supplying carbon dioxide and hydrogen to a reaction device accommodating a catalyst which promotes a methanation reaction, and causing the carbon dioxide and the hydrogen to react with each other in the reaction device.

### CITATION LIST

### Patent Literature

Patent Literature 1: JP 2021-116294 A

### SUMMARY

### Technical Problem

A synthesis reaction of hydrogen with any one or both of carbon dioxide and carbon monoxide, such as a methanation reaction, is an exothermic reaction. Thus, in a technology of promoting the synthesis reaction between carbon dioxide and hydrogen through use of a catalyst as in Patent Literature 1 above, there is a risk in that the temperature inside the reaction device may be raised excessively due to the reaction heat of the synthesis reaction, resulting in that the catalyst may deteriorate.

In view of such problem, an object of the present disclosure is to provide a hydrocarbon production apparatus capable of setting the inside of a reaction device to an appropriate temperature.

### Solution to Problem

In order to solve the problem described above, according to one aspect of the present disclosure, there is provided a hydrocarbon production apparatus, including: a reaction device, which has an inlet, an outlet, and one or a plurality of supply ports formed between the inlet and the outlet, and in which a catalyst that promotes an exothermic reaction between hydrogen and carbon dioxide is accommodated; a first hydrogen supply unit that supplies the hydrogen into the reaction device through the inlet of the reaction device; and a carbon dioxide supply unit that supplies the carbon dioxide into the reaction device through the inlet and the one or the plurality of supply ports of the reaction device.

Further, the above-mentioned hydrocarbon production apparatus may further include a flow rate control unit that controls a flow rate of the carbon dioxide supplied by the carbon dioxide supply unit.

Further, the flow rate control unit may control the flow rate of the carbon dioxide based on a temperature of the catalyst accommodated in the reaction device.

Further, a distance between the inlet and the supply port having a shortest distance from the inlet may be shorter than a distance between the outlet and the supply port having a shortest distance from the outlet.

Further, the reaction device may have two or more supply ports, the distance between the inlet and the supply port having a shortest distance from the inlet may be shorter than a distance between the supply ports adjacent to each other, and the distance between the outlet and the supply port having a shortest distance from the outlet may be longer than a distance between the supply ports adjacent to each other.

Further, the reaction device may have three or more supply ports, and the distance between the supply ports adjacent to each other may increase from the inlet side toward the outlet side.

Further, the reaction device may include a plurality of reactors connected in series, the inlet may be formed in the reactor at a foremost stage, and the outlet may be formed in the reactor at a rearmost stage.

Further, the above-mentioned hydrocarbon production apparatus may further include a cooler that cools a gas discharged from a discharge port of at least any one of the plurality of reactors.

Further, the above-mentioned hydrocarbon production apparatus may further include a second hydrogen supply unit that supplies the hydrogen into the reaction device through the one or the plurality of supply ports of the reaction device.

In order to solve the problem described above, according to one aspect of the present disclosure, there is provided another hydrocarbon production apparatus, including: a reaction device, which has an inlet, an outlet, and one or a plurality of supply ports formed between the inlet and the outlet, and in which a catalyst that promotes an exothermic reaction between hydrogen and carbon monoxide is accommodated; a first hydrogen supply unit that supplies the hydrogen into the reaction device through the inlet of the reaction device; and a carbon monoxide supply unit that supplies the carbon monoxide into the reaction device through the inlet and the one or the plurality of supply ports of the reaction device.

In order to solve the problem described above, according to one aspect of the present disclosure, there is provided still another hydrocarbon production apparatus, including: a reaction device, which has an inlet, an outlet, and one or a plurality of supply ports formed between the inlet and the outlet, and in which a catalyst that promotes an exothermic reaction among hydrogen, carbon monoxide, and carbon dioxide is accommodated; a first hydrogen supply unit that supplies the hydrogen into the reaction device through the inlet of the reaction device; a carbon monoxide supply unit that supplies the carbon monoxide into the reaction device through the inlet and the one or the plurality of supply ports of the reaction device; and a carbon dioxide supply unit that supplies the carbon dioxide into the reaction device through the inlet and the one or the plurality of supply ports of the reaction device.

### Effects

According to the present disclosure, it is possible to set the inside of the reaction device to an appropriate temperature.

### BRIEF DESCRIPTION OF DRAWINGS

FIG. 1 is an explanatory diagram for illustrating a hydrocarbon production apparatus according to a first embodiment.

FIG. 2 is an explanatory diagram for illustrating a hydrocarbon production apparatus according to a second embodiment.

FIG. 3 is an explanatory diagram for illustrating a hydrocarbon production apparatus according to a modification example.

FIG. 4 is a graph for showing simulation results of Comparative Example.

FIG. 5 is a graph for showing simulation results of a first reactor in Example.

FIG. 6 is a graph for showing simulation results of a second reactor in Example.

### DESCRIPTION OF EMBODIMENTS

Now, with reference to the attached drawings, embodiments of the present disclosure are described in detail. The dimensions, materials, and other specific numerical values represented in the embodiments are merely examples used for facilitating the understanding of the disclosure, and do not limit the present disclosure unless otherwise particularly noted. Elements having substantially the same functions and configurations herein and in the drawings are denoted by the same reference symbols to omit redundant description thereof. Further, illustration of elements with no direct relationship to the present disclosure is omitted.

### First Embodiment: Hydrocarbon Production Apparatus 100

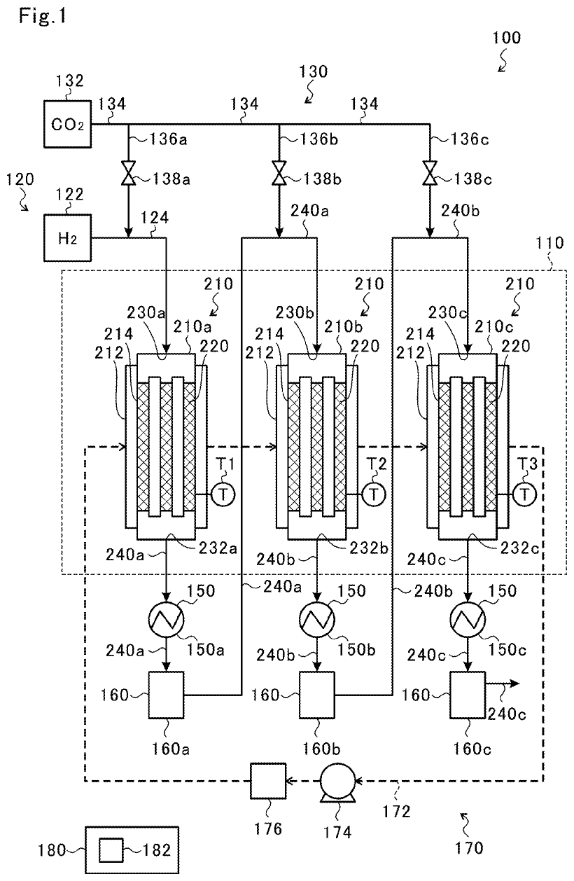

FIG. 1 is an explanatory diagram for illustrating a hydrocarbon production apparatus 100 according to a first embodiment. As illustrated in FIG. 1, the hydrocarbon production apparatus 100 according to the first embodiment includes a reaction device 110, temperature sensors T1, T2, and T3, a hydrogen supply unit 120, a carbon dioxide supply unit 130, a cooler 150, a gas-liquid separator 160, a temperature adjustment unit 170, and a central controller 180. In FIG. 1, the arrows in the solid lines indicate flows of hydrogen (gas), carbon dioxide (gas), a hydrocarbon (gas), and water (gas and liquid). Moreover, in FIG. 1, the arrows in the broken lines indicate flows of heat media.

In the reaction device 110, a synthesis reaction between hydrogen and carbon dioxide takes place, resulting in the production of the hydrocarbon. The synthesis reaction between hydrogen and carbon dioxide is an exothermic reaction. For example, the synthesis reaction between hydrogen and carbon dioxide is a reaction represented by formulae (1) to (4) below.

4H₂+CO₂→CH₄+2H₂O Formula (1)

6H₂+2CO₂→C₂H₄+4H₂O Formula (2)

9H₂+3CO₂→C₃H₆+6H₂O Formula (3)

mH₂+nCO₂→hydrocarbon+2nH₂O Formula (4)

Methane is produced through the reaction represented by the formula (1). Ethylene is produced through the reaction represented by the formula (2). Propylene is produced through the reaction represented by the formula (3). A hydrocarbon is produced through the Fischer-Tropsch (FT) synthesis reaction represented by the formula (4). In this embodiment, there is exemplified a case in which the reaction represented by the formula (1) takes place in the reaction device 110, to thereby produce methane.

The reaction device 110 has an inlet 230a for hydrogen and carbon dioxide, an outlet 232c for a reacted gas, and one or a plurality of supply ports 230b and 230c for carbon dioxide formed between the inlet 230a and the outlet 232c. In addition, a catalyst 220 is accommodated in the reaction device 110. A layer of the catalyst 220 (catalyst layer) is formed in the reaction device 110. The catalyst 220 is a catalyst that promotes the exothermic reaction between hydrogen and carbon dioxide. The catalyst 220 is, for example, a catalyst that promotes the reaction represented by the formula (1). In FIG. 1, the catalyst 220 is illustrated by cross-hatching.

In this embodiment, the reaction device 110 includes, for example, a plurality of reactors 210 connected in series. The reaction device 110 is formed of a plurality of reactors 210 connected in series, and hence the supply ports 230b and 230c for carbon dioxide can be easily formed. The reactor 210 is, for example, a multi-tube type and heat-exchange-type reactor. The reactor 210 includes a heat medium container 212 and a plurality of reaction tubes 214. To the heat medium container 212, the heat medium is supplied by the temperature adjustment device 170 described later.

The plurality of reaction tubes 214 are placed in the heat medium container 212. The catalyst 220 is accommodated in the reaction tube 214. A layer of the catalyst 220 (catalyst layer) is formed in the reaction tube 214. The temperature in the reaction tube 214 (temperature of the catalyst layer) becomes, for example, 200° C. or more and 550° C. or less due to the heat medium passing through the heat medium container 212 and the heat generated by the exothermic reaction that proceeds in the reaction tube 214.

In this embodiment, the reaction device 110 is formed of three reactors 210, for example, a first reactor 210a, a second reactor 210b, and a third reactor 210c. The first reactor 210a, the second reactor 210b, and the third reactor 210c are connected in series in the stated order.

The inlet 230a and a discharge port 232a are formed in the first reactor 210a at a foremost stage. The inlet 230a is in communication with one side of the plurality of reaction tubes 214 of the first reactor 210a. The discharge port 232a is in communication with another side of the plurality of reaction tubes 214 of the first reactor 210a. The hydrogen supply unit 120 and the carbon dioxide supply unit 130 described later are connected to the inlet 230a of the first reactor 210a. The supply port 230b of the second reactor 210b is connected to the discharge port 232a of the first reactor 210a through a piping 240a.

The supply port 230b and a discharge port 232b are formed in the second reactor 210b. The supply port 230b is in communication with one side of the plurality of reaction tubes 214 of the second reactor 210b. The discharge port 232b is in communication with another side of the plurality of reaction tubes 214 of the second reactor 210b. The supply port 230c of the third reactor 210c at a rearmost stage is connected to the discharge port 232b of the second reactor 210b through a piping 240b.

The supply port 230c and the outlet 232c are formed in the third reactor 210c. The supply port 230c is in communication with one side of the plurality of reaction tubes 214 of the third reactor 210c. The outlet 232c is in communication with another side of the plurality of reaction tubes 214 of the third reactor 210c. A piping 240c is connected to the outlet 232c of the third reactor 210c. A generated gas produced in the reaction device 110, which contains 90% or more hydrocarbon, is discharged to the outside through the piping 240c.

In this embodiment, for example, the length of the reaction tube 214 of the reactor 210 increases from the foremost stage to the rearmost stage. For example, the length of the reaction tube 214 may increase in the order of the first reactor 210a, the second reactor 210b, and the third reactor 210c. That is, in the reaction device 110, the distance between the inlet 230a and the supply port 230b having a shortest distance from the inlet 230a may be shorter than the distance between the supply port 230b and the supply port 230c. In addition, in the reaction device 110, the distance between the outlet 232c and the supply port 230c having a shortest distance from the outlet 232c may be longer than the distance between the supply port 230b and the supply port 230c.

The temperature sensor T1 detects the temperature of the catalyst 220 (catalyst layer) accommodated in the reaction tube 214 of the first reactor 210a. The temperature sensor T2 detects the temperature of the catalyst 220 (catalyst layer) accommodated in the reaction tube 214 of the second reactor 210b. The temperature sensor T3 detects the temperature of the catalyst 220 (catalyst layer) accommodated in the reaction tube 214 of the third reactor 210c.

The hydrogen supply unit 120 (first hydrogen supply unit) supplies hydrogen into the first reactor 210a through the inlet 230a of the first reactor 210a of the reaction device 110.

The hydrogen supply unit 120 includes, for example, a blower 122 and a raw gas supply piping 124. A suction side of the blower 122 is connected to a supply source of hydrogen. A discharge side of the blower 122 is connected to the raw gas supply piping 124. The raw gas supply piping 124 connects the discharge side of the blower 122 to the inlet 230a of the first reactor 210a of the reaction device 110.

The carbon dioxide supply unit 130 supplies carbon dioxide into the first reactor 210a, into the second reactor 210b, and into the third reactor 210c through the inlet 230a of the first reactor 210a, the supply port 230b of the second reactor 210b, and the supply port 230c of the third reactor 210c of the reaction device 110.

The carbon dioxide supply unit 130 includes, for example, a blower 132, a carbon dioxide supply piping 134, branch piping 136a, 136b, and 136c, and flow rate adjustment valves 138a, 138b, and 138c. A suction side of the blower 132 is connected to a supply source of carbon dioxide. A discharge side of the blower 132 is connected to the carbon dioxide supply piping 134.

The branch piping 136a connects the carbon dioxide supply piping 134 to the raw gas supply piping 124. Carbon dioxide discharged from the blower 132 is supplied to the first reactor 210a through the carbon dioxide supply piping 134, the branch piping 136a, the raw gas supply piping 124, and the inlet 230a of the first reactor 210a of the reaction device 110.

The branch piping 136b connects the carbon dioxide supply piping 134 to the piping 240a. Carbon dioxide discharged from the blower 132 is supplied to the second reactor 210b through the carbon dioxide supply piping 134, the branch piping 136b, the piping 240a, and the supply port 230b of the second reactor 210b of the reaction device 110.

The branch piping 136c connects the carbon dioxide supply piping 134 to the piping 240b. Carbon dioxide discharged from the blower 132 is supplied to the third reactor 210c through the carbon dioxide supply piping 134, the branch piping 136c, the piping 240b, and the supply port 230c of the third reactor 210c of the reaction device 110.

The flow rate adjustment valve 138a is provided to the branch piping 136a. The flow rate adjustment valve 138a adjusts the opening degree of a flow passage formed in the branch piping 136a. The flow rate adjustment valve 138b is provided to the branch piping 136b. The flow rate adjustment valve 138b adjusts the opening degree of a flow passage formed in the branch piping 136b. The flow rate adjustment valve 138c is provided to the branch piping 136c. The flow rate adjustment valve 138c adjusts the opening degree of a flow passage formed in the branch piping 136c. The flow rate adjustment valves 138a, 138b, and 138c have their opening degrees adjusted by a flow rate control unit 182 described later. The opening degree adjustment process performed by the flow rate control unit 182 is described in detail later.

The cooler 150 cools a gas discharged from the discharge ports 232a and 232b of at least any one of the plurality of reactors 210. The cooler 150 is, for example, a heat exchanger. In this embodiment, the hydrocarbon production apparatus 100 includes, for example, three coolers 150a, 150b, and 150c.

The cooler 150a is provided to the piping 240a. The cooler 150a cools a gas discharged from the discharge port 232a of the first reactor 210a. The gas discharged from the first reactor 210a contains hydrogen, carbon dioxide, methane, and water (gas). The cooler 150b is provided to the piping 240b. The cooler 150b cools a gas discharged from the discharge port 232b of the second reactor 210b. The gas discharged from the second reactor 210b contains hydrogen, carbon dioxide, methane, and water (gas). The cooler 150c is provided to the piping 240c. The cooler 150c cools a gas discharged from the outlet 232c of the third reactor 210c. The gas discharged from the third reactor 210c contains hydrogen, carbon dioxide, methane, and water (gas).

The gas-liquid separator 160 separates a mixture of the gas cooled by the cooler 150 and a liquid (liquid water) into a gas and a liquid. In this embodiment, the hydrocarbon production apparatus 100 includes, for example, three gas-liquid separators 160a, 160b, and 160c.

The gas-liquid separator 160a is provided to the piping 240a. The gas-liquid separator 160a separates a mixture of the gas cooled by the cooler 150a and liquid water into a gas and a liquid. The gas-liquid separator 160b is provided to the piping 240b. The gas-liquid separator 160b separates a mixture of the gas cooled by the cooler 150b and liquid water into a gas and a liquid. The gas-liquid separator 160c is provided to the piping 240c. The gas-liquid separator 160c separates a mixture of the gas cooled by the cooler 150c and liquid water into a gas and a liquid.

The temperature adjustment unit 170 maintains the temperature of the reactor 210 of the reaction device 110 within a predetermined reaction temperature range. The reaction temperature range is the range of temperatures at which the synthesis reaction between hydrogen and carbon dioxide efficiently proceeds and at which the deterioration of the catalyst 220 can be suppressed. The reaction temperature range is, for example, 200° C. or more and 550° C. or less, preferably, 300° C. or more and 500° C. or less.

The temperature adjustment unit 170, for example, circulates a heat medium at a predetermined temperature in the heat medium container 212 of the first reactor 210a, the heat medium container 212 of the second reactor 210b, and the heat medium container 212 of the third reactor 210c. The temperature adjustment unit 170 includes, for example, a circulation path 172, a pump 174, and a heating and cooling unit 176. The circulation path 172 is a flow passage through which the heat medium circulates. The pump 174, the heating and cooling unit 176, the heat medium container 212 of the first reactor 210a, the heat medium container 212 of the second reactor 210b, and the heat medium container 212 of the third reactor 210c are placed in the circulation path 172 in the stated order. A suction side of the pump 174 is connected to the heat medium container 212 of the third reactor 210c. A discharge side of the pump 174 is connected to the heating and cooling unit 176. When the pump 174 is operated, the heat medium circulates through the circulation path 172. The heating and cooling unit 176 heats or cools the heat medium.

The central controller 180 is formed of a semiconductor integrated circuit including a central processing unit (CPU). The central controller 180 reads out, for example, a program and a parameter each for operating the CPU from a ROM. The central controller 180 manages and controls the entire hydrocarbon production apparatus 100 in cooperation with a RAM serving as a working area and other electronic circuit.

In this embodiment, the central controller 180 controls the temperature adjustment unit 170 to circulate the heat medium to the first reactor 210a, the second reactor 210b, and the third reactor 210c. In addition, the central controller 180 controls the hydrogen supply unit 120 to supply a predetermined amount of hydrogen to the reaction device 110. The central controller 180 controls the carbon dioxide supply unit 130 to supply a predetermined amount of carbon dioxide to the reaction device 110.

In addition, the central controller 180 functions, for example, as the flow rate control unit 182. The flow rate control unit 182 controls the flow rate of carbon dioxide supplied by the carbon dioxide supply unit 130 to the first reactor 210a, the second reactor 210b, and the third reactor 210c, respectively, for example, based on the detected values of the temperature sensors T1, T2, and T3. In this embodiment, the flow rate control unit 182 controls the flow rate of carbon dioxide supplied to each of the first reactor 210a, the second reactor 210b, and the third reactor 210c by adjusting the opening degrees of the flow rate adjustment valves 138a, 138b, and 138c, respectively.

In this embodiment, the flow rate control unit 182 adjusts the opening degrees of the flow rate adjustment valves 138a, 138b, and 138c, respectively, such that the detected value of the temperature sensor T1 (temperature of the first reactor 210a), the detected value of the temperature sensor T2 (temperature of the second reactor 210b), and the detected value of the temperature sensor T3 (temperature of the third reactor 210c) all fall within the above-mentioned reaction temperature range, for example, 200° C. or more and 550° C. or less. For example, when the detected value of the temperature sensor T1 (temperature of the first reactor 210a) is higher than the reaction temperature range, the flow rate control unit 182 decreases the opening degree of the flow rate adjustment valve 138a. In addition, for example, when the detected value of the temperature sensor T1 (temperature of the first reactor 210a) is lower than the reaction temperature range, the flow rate control unit 182 increases the opening degree of the flow rate adjustment valve 138a.

As described above, the hydrocarbon production apparatus 100 according to this embodiment includes the first reactor 210a, the second reactor 210b, the third reactor 210c, the hydrogen supply unit 120 that supplies hydrogen to the first reactor 210a, and the carbon dioxide supply unit 130 that supplies carbon dioxide to the first reactor 210a, the second reactor 210b, and the third reactor 210c.

In the related art in which hydrogen and carbon dioxide are supplied only to the first reactor 210a at the foremost stage, the exothermic reaction proceeds rapidly in the first reactor 210a, and the temperature of the first reactor 210a rises excessively. Thus, in the related art, there is a problem in that the catalyst 220 in the first reactor 210a deteriorates due to heat.

In contrast, the hydrocarbon production apparatus 100 according to this embodiment supplies carbon dioxide in a divided manner to each of the plurality of reactors 210, and hence can suppress rapid progress of the exothermic reaction in the first reactor 210a and can avoid a situation in which the temperature of the first reactor 210a rises excessively. With this configuration, the hydrocarbon production apparatus 100 can set the inside of the reaction device 110 to an appropriate temperature. Thus, the hydrocarbon production apparatus 100 can prevent deterioration of the catalyst 220 in the reaction device 110 due to heat.

In addition, it is also conceivable to use a comparative technology in which carbon dioxide is supplied only to the first reactor 210a at the foremost stage and hydrogen is divided and supplied to the first reactor 210a, the second reactor 210b, and the third reactor 210c. However, in the comparative technology, the amount of carbon dioxide supplied to the first reactor 210a is larger than that in the hydrocarbon production apparatus 100 according to this embodiment. In that case, there is a problem in that the catalyst 220 in the first reactor 210a undergoes oxidative deterioration due to the carbon dioxide.

In contrast, the hydrocarbon production apparatus 100 according to this embodiment supplies hydrogen only to the first reactor 210a at the foremost stage, and supplies carbon dioxide in a divided manner to each of the first reactor 210a, the second reactor 210b, and the third reactor 210c, and hence can decrease the amount of carbon dioxide supplied to the first reactor 210a as compared to the comparative technology. In addition, the hydrocarbon production apparatus 100 according to this embodiment can increase the amount of hydrogen supplied to the first reactor 210a as compared to the comparative technology. With this configuration, the hydrocarbon production apparatus 100 can avoid a situation in which the catalyst 220 in the first reactor 210a undergoes oxidative deterioration.

In addition, as represented by the formulae (1) to (4), in the synthesis reaction of hydrogen and carbon dioxide to hydrocarbon, the number of moles (flow rate) of hydrogen is larger than that of carbon dioxide in the stoichiometric ratio. Thus, the hydrocarbon production apparatus 100 according to this embodiment supplies carbon dioxide having a relatively small flow rate in a divided manner to each of the reactors 210, and by adjusting the flow rate of carbon dioxide supplied to each of the reactors 210, can easily control the temperature of each of the reactors 210. In addition, the hydrocarbon production apparatus 100 according to this embodiment supplies the entire amount of hydrogen having a relatively large flow rate to the first reactor 210a, and hence can increase the amount of a gas that does not contribute to the reaction in the first reactor 210a as compared to the comparative technology. With this configuration, the hydrocarbon production apparatus 100 according to this embodiment can suppress the increase in reaction heat due to the gas that does not contribute to the reaction as compared to the comparative technology.

In addition, as described above, the flow rate control unit 182 controls the flow rate of carbon dioxide based on the temperature of the catalyst 220 accommodated in the reaction device 110. With this configuration, the hydrocarbon production apparatus 100 can set the temperature of the first reactor 210a, the temperature of the second reactor 210b, and the temperature of the third reactor 210c all within the reaction temperature range. Thus, the hydrocarbon production apparatus 100 can reduce activation time, prevent further thermal deterioration of the catalyst 220, and prevent a decrease in hydrocarbon production efficiency.

More specifically, in an activation process for activating the hydrocarbon production apparatus 100, for example, the flow rate control unit 182 adjusts the opening degree of the flow rate adjustment valve 138a such that the stoichiometric ratio between the hydrogen supplied from the hydrogen supply unit 120 to the first reactor 210a and the carbon dioxide supplied from the carbon dioxide supply unit 130 to the first reactor 210a satisfies the formula (1). In addition, the flow rate control unit 182 adjusts the opening degree of the flow rate adjustment valve 138b such that the stoichiometric ratio between the remaining hydrogen supplied from the first reactor 210a to the second reactor 210b and the carbon dioxide supplied from the carbon dioxide supply unit 130 to the second reactor 210b satisfies the formula (1). In addition, the flow rate control unit 182 adjusts the opening degree of the flow rate adjustment valve 138c such that the stoichiometric ratio between the remaining hydrogen supplied from the second reactor 210b to the third reactor 210c and the carbon dioxide supplied from the carbon dioxide supply unit 130 to the third reactor 210c satisfies the formula (1).

In this manner, the flow rate control unit 182 supplies carbon dioxide to each of the first reactor 210a, the second reactor 210b, and the third reactor 210c such that the stoichiometric ratio is achieved in the activation process, and as a result, the exothermic reaction represented by the formula (1) is allowed to proceed efficiently in the first reactor 210a, the second reactor 210b, and the third reactor 210c. Thus, the hydrocarbon production apparatus 100 can rapidly raise the temperatures of the first reactor 210a, the second reactor 210b, and the third reactor 210c.

Meanwhile, in the related art in which carbon dioxide is supplied only to the first reactor 210a, the exothermic reaction represented by the formula (1) proceeds less in the second reactor 210b and the third reactor 210c than in the first reactor 210a, resulting in lower temperature rise rates of the second reactor 210b and the third reactor 210c as compared to the first reactor 210a. Thus, in the related art in which carbon dioxide is supplied only to the first reactor 210a, there is a problem in that the activation time of the first reactor 210a, the second reactor 210b, and the third reactor 210c is prolonged.

In contrast, the hydrocarbon production apparatus 100 can rapidly raise the temperatures of the first reactor 210a, the second reactor 210b, and the third reactor 210c, and hence the activation time of the first reactor 210a, the second reactor 210b, and the third reactor 210c can be reduced as compared to the related art. In addition, the hydrocarbon production apparatus 100 can eliminate the need for a heating mechanism for activation that heats the second reactor 210b and the third reactor 210c, thereby being capable of reducing the overall cost of the apparatus.

In addition, in a normal operation process for normally operating the hydrocarbon production apparatus 100, for example, the flow rate control unit 182 adjusts the opening degrees of the flow rate adjustment valves 138a, 138b, and 138c such that the stoichiometric ratio between the hydrogen supplied from the hydrogen supply unit 120 to the first reactor 210a and the total of the carbon dioxide supplied from the carbon dioxide supply unit 130 to the first reactor 210a, the second reactor 210b, and the third reactor 210c satisfies the formula (1). In addition, for example, the flow rate control unit 182 adjusts the opening degrees of the flow rate adjustment valves 138a, 138b, and 138c such that the supply amount of carbon dioxide increases in the order of the first reactor 210a, the second reactor 210b, and the third reactor 210c.

For example, the flow rate control unit 182 adjusts the opening degrees of the flow rate adjustment valves 138a and 138b such that the amount of the carbon dioxide supplied from the carbon dioxide supply unit 130 to the first reactor 210a is smaller than that of the carbon dioxide supplied from the carbon dioxide supply unit 130 to the second reactor 210b. In addition, for example, the flow rate control unit 182 adjusts the opening degrees of the flow rate adjustment valves 138b and 138c such that the amount of the carbon dioxide supplied from the carbon dioxide supply unit 130 to the second reactor 210b is smaller than that of the carbon dioxide supplied from the carbon dioxide supply unit 130 to the third reactor 210c.

With this configuration, carbon dioxide in an amount smaller than the stoichiometric ratio is supplied to the first reactor 210a and the second reactor 210b. Thus, the rapid progress of the synthesis reaction represented by the formula (1) can be prevented in the first reactor 210a and the second reactor 210b. As described above, the synthesis reaction represented by the formula (1) is an exothermic reaction, and hence with the prevention of the rapid progress of the synthesis reaction represented by the formula (1), a situation in which the temperatures of the first reactor 210a and the second reactor 210b rise excessively can be avoided. Thus, the hydrocarbon production apparatus 100 can avoid a situation in which the catalyst 220 deteriorates due to heat in the first reactor 210a and the second reactor 210b. In addition, the hydrocarbon production apparatus 100 can eliminate the need for a dedicated cooling mechanism for cooling the first reactor 210a and the second reactor 210b, thereby being capable of reducing the overall cost of the apparatus.

In addition, when the supply source of hydrogen is a water electrolysis device that performs electrolysis of water by using renewable energy such as solar power generation, the supply amount of hydrogen by the hydrogen supply unit 120 may fluctuate. In this case, there is a risk in that any one or the plurality of the reactors 210 among the first reactor 210a, the second reactor 210b, and the third reactor 210c may fall below the reaction temperature range. In addition, the temperature of the heat medium circulating through the first reactor 210a, the second reactor 210b, and the third reactor 210c may rapidly fluctuate or the heat medium may flow unevenly. In this case as well, there is a risk in that any one or the plurality of the reactors 210 among the first reactor 210a, the second reactor 210b, and the third reactor 210c may fall below the reaction temperature range.

In view of the foregoing, in a load fluctuation process in which the supply amount of hydrogen fluctuates or the heat medium causes a malfunction, for example, the flow rate control unit 182 adjusts the opening degrees of the flow rate adjustment valves 138a, 138b, and 138c so as to increase the supply amount of carbon dioxide to the reactor 210 in which the temperature of the catalyst 220 has fallen below the reaction temperature range. With this configuration, the hydrocarbon production apparatus 100 can raise the temperature of the reactor 210 in which the temperature of the catalyst 220 has fallen below the reaction temperature range. Thus, the hydrocarbon production apparatus 100 can prevent a decrease in hydrocarbon production efficiency due to a decrease in temperature.

In addition, as described above, in the reaction device 110, the distance between the inlet 230a and the supply port 230b having a shortest distance from the inlet 230a may be shorter than the distance between the supply port 230b and the supply port 230c. That is, in the reaction device 110, the distance between the supply port 230b and the supply port 230c may be longer than the distance between the inlet 230a and the supply port 230b having a shortest distance from the inlet 230a. That is, the length of the reaction tube 214 of the second reactor 210b may be longer than that of the first reactor 210a. With this configuration, the residence time of the gas in the second reactor 210b can be set to be longer than that in the first reactor 210a. Thus, the conversion rate of carbon dioxide in the second reactor 210b can be improved. The phrase “conversion rate of carbon dioxide” refers to a ratio at which carbon dioxide is converted into hydrocarbon.

In addition, as described above, in the reaction device 110, the distance between the outlet 232c and the supply port 230c having a shortest distance from the outlet 232c may be longer than the distance between the supply port 230b and the supply port 230c. That is, the length of the reaction tube 214 of the third reactor 210c may be longer than that of the second reactor 210b. With this configuration, the residence time of the gas in the third reactor 210c can be set to be longer than that in the second reactor 210b. Thus, the conversion rate of carbon dioxide in the third reactor 210c can be improved.

In addition, as described above, the hydrocarbon production apparatus 100 includes the cooler 150. The synthesis reaction represented by the formulae (1) to (4) is an exothermic reaction, and hence the reaction is more likely to proceed at a low temperature than at a high temperature (the conversion rate of carbon dioxide is higher). Thus, with the provision of the cooler 150, the hydrocarbon production apparatus 100 can further promote the progress of the synthesis reaction represented by the formulae (1) to (4) in the second reactor 210b and the third reactor 210c.

In addition, as described above, the hydrocarbon production apparatus 100 includes the gas-liquid separator 160. In the synthesis reaction represented by the formulae (1) to (4), water is generated. Thus, based on the equilibrium relationship, the removal of water from the generated gas can increase the conversion rate of carbon dioxide. Thus, with the provision of the gas-liquid separator 160, the hydrocarbon production apparatus 100 can further promote the progress of the synthesis reaction represented by the formulae (1) to (4) in the second reactor 210b and the third reactor 210c.

### Second Embodiment: Hydrocarbon Production Apparatus 300

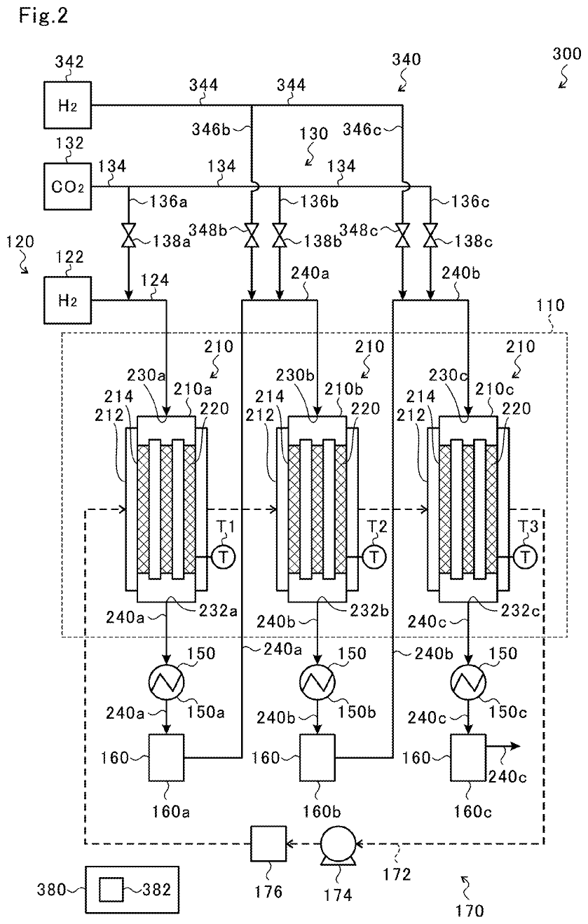

FIG. 2 is an explanatory diagram for illustrating a hydrocarbon production apparatus 300 according to a second embodiment. As illustrated in FIG. 2, the hydrocarbon production apparatus 300 according to the second embodiment includes a reaction device 110, a hydrogen supply unit 120, a carbon dioxide supply unit 130, a hydrogen supply unit 340, a cooler 150, a gas-liquid separator 160, a temperature adjustment unit 170, and a central controller 380. In FIG. 2, the arrows in the solid lines indicate flows of hydrogen (gas), carbon dioxide (gas), a hydrocarbon (gas), and water (gas and liquid). Moreover, in FIG. 2, the arrows in the broken lines indicate flows of heat media. Elements that are substantially equivalent to those of the hydrocarbon production apparatus 100 are denoted by the same reference symbols, and description thereof is omitted.

The hydrogen supply unit 340 (second hydrogen supply unit) supplies hydrogen into the reaction device 110 through the supply ports 230b and 230c of the reaction device 110. That is, the hydrogen supply unit 340 supplies hydrogen into the second reactor 210b through the supply port 230b of the second reactor 210b and into the third reactor 210c through the supply port 230c of the third reactor 210c.

The hydrogen supply unit 340 includes, for example, a blower 342, a hydrogen supply piping 344, branch piping 346b and 346c, and flow rate adjustment valves 348b and 348c. A suction side of the blower 342 is connected to a supply source of hydrogen. A discharge side of the blower 342 is connected to the hydrogen supply piping 344.

The branch piping 346b connects the hydrogen supply piping 344 to the piping 240a. Hydrogen discharged from the blower 342 is supplied to the second reactor 210b through the hydrogen supply piping 344, the branch piping 346b, the piping 240a, and the supply port 230b of the second reactor 210b of the reaction device 110.

The branch piping 346c connects the hydrogen supply piping 344 to the piping 240b. Hydrogen discharged from the blower 342 is supplied to the third reactor 210c through the hydrogen supply piping 344, the branch piping 346c, the piping 240b, and the supply port 230c of the third reactor 210c of the reaction device 110.

The flow rate adjustment valve 348b is provided to the branch piping 346b. The flow rate adjustment valve 348b adjusts the opening degree of a flow passage formed in the branch piping 346b. The flow rate adjustment valve 348c is provided to the branch piping 346c. The flow rate adjustment valve 348c adjusts the opening degree of a flow passage formed in the branch piping 346c. The flow rate adjustment valves 348b and 348c have their opening degrees adjusted by a flow rate control unit 382 described later.

The central controller 380 is formed of a semiconductor integrated circuit including a central processing unit (CPU). The central controller 380 reads out, for example, a program and a parameter each for operating the CPU from a ROM. The central controller 380 manages and controls the entire hydrocarbon production apparatus 300 in cooperation with a RAM serving as a working area and other electronic circuit.

In this embodiment, in the same manner as in the above-mentioned first embodiment, the central controller 380 controls the temperature adjustment unit 170 to circulate the heat medium to the first reactor 210a, the second reactor 210b, and the third reactor 210c. In addition, the central controller 380 controls the hydrogen supply unit 120 to supply a predetermined amount of hydrogen to the reaction device 110. The central controller 380 controls the carbon dioxide supply unit 130 to supply a predetermined amount of carbon dioxide to the reaction device 110.

In addition, the central controller 380 functions, for example, as the flow rate control unit 382. The flow rate control unit 382 controls the flow rate of carbon dioxide supplied by the carbon dioxide supply unit 130 to the first reactor 210a, the second reactor 210b, and the third reactor 210c, respectively, and the flow rate of hydrogen supplied by the hydrogen supply unit 340 to the first reactor 210a, the second reactor 210b, and the third reactor 210c, respectively, for example, based on the detected values of the temperature sensors T1, T2, and T3. In this embodiment, the flow rate control unit 382 controls the flow rate of carbon dioxide and the flow rate of hydrogen supplied to each of the first reactor 210a, the second reactor 210b, and the third reactor 210c by adjusting the opening degrees of the flow rate adjustment valves 138a, 138b, 138c, 348b, and 348c, respectively.

In this embodiment, the flow rate control unit 382 adjusts the opening degrees of the flow rate adjustment valves 138a, 138b, 138c, 348b, and 348c, respectively, such that the detected value of the temperature sensor T1 (temperature of the first reactor 210a), the detected value of the temperature sensor T2 (temperature of the second reactor 210b), and the detected value of the temperature sensor T3 (temperature of the third reactor 210c) all fall within the reaction temperature range.

As described above, the hydrocarbon production apparatus 300 according to this embodiment includes the hydrogen supply unit 340 in addition to the carbon dioxide supply unit 130, and the flow rate control unit 382 controls the flow rate of carbon dioxide supplied by the carbon dioxide supply unit 130 to the first reactor 210a, the second reactor 210b, and the third reactor 210c, respectively, and the flow rate of hydrogen supplied by the hydrogen supply unit 340 to the first reactor 210a, the second reactor 210b, and the third reactor 210c, respectively, based on the detected values of the temperature sensors T1, T2, and T3.

With this configuration, the hydrocarbon production apparatus 300 according to this embodiment can control the temperatures of the first reactor 210a, the second reactor 210b, and the third reactor 210c with higher accuracy.

### Modification Example: Hydrocarbon Production Apparatus 400

In the above-mentioned first embodiment, there has been given, as an example, the case in which the reaction device 110 includes the plurality of reactors 210. However, the reaction device 110 may include only one reactor.

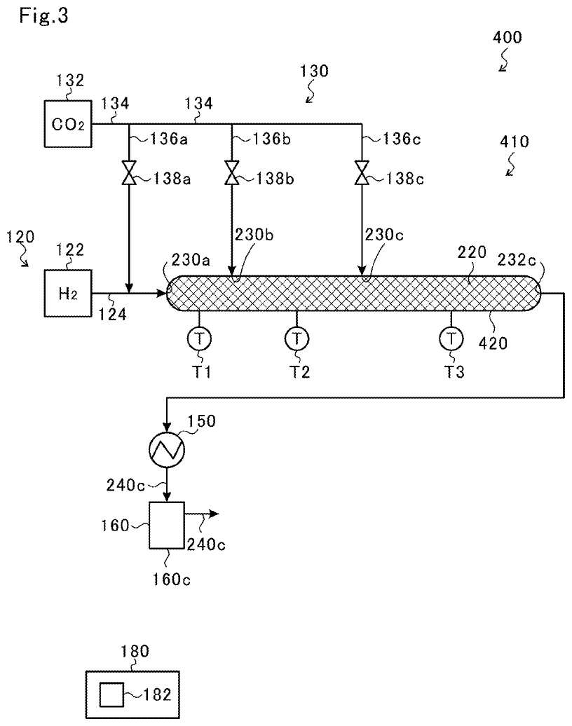

FIG. 3 is an explanatory diagram for illustrating a hydrocarbon production apparatus 400 according to a modification example. As illustrated in FIG. 3, the hydrocarbon production apparatus 400 according to the modification example includes a reaction device 410, a hydrogen supply unit 120, a carbon dioxide supply unit 130, a cooler 150, a gas-liquid separator 160, a temperature adjustment unit 170, and a central controller 180. In FIG. 3, the arrows in the solid lines indicate flows of hydrogen (gas), carbon dioxide (gas), a hydrocarbon (gas), and water (gas and liquid). Elements that are substantially equivalent to those of the hydrocarbon production apparatus 100 are denoted by the same reference symbols, and description thereof is omitted.

In this embodiment, the reaction device 410 includes one reactor 420 and the temperature sensors T1, T2, and T3.

The reactor 420 is, for example, an adiabatic reactor. In the reactor 420, the inlet 230a, the supply ports 230b and 230c, and the outlet 232c are formed in the stated order. In addition, in the same manner as in the above-mentioned first embodiment, the catalyst 220 is accommodated in the reactor 420.

In the modification example, the temperature sensor T1 detects the temperature of a catalyst layer accommodated between the inlet 230a and the supply port 230b of the reactor 420. The temperature sensor T2 detects the temperature of a catalyst layer accommodated between the supply port 230b and the supply port 230c of the reactor 420. The temperature sensor T3 detects the temperature of a catalyst layer accommodated between the supply port 230c and the outlet 232c of the reactor 420.

In the hydrocarbon production apparatus 400 according to the modification example as well, carbon dioxide is supplied in a divided manner to each of the inlet 230a and the supply ports 230b and 230c, and hence the rapid progress of the exothermic reaction can be suppressed in the reactor 420 and a situation in which the temperature rises excessively in the reactor 420 can be avoided. With this configuration, the hydrocarbon production apparatus 400 can set the inside of the reaction device 410 to an appropriate temperature. Thus, the hydrocarbon production apparatus 400 can prevent deterioration of the catalyst 220 in the reaction device 410 due to heat.

### EXAMPLE

As Example, the temperature of a catalyst layer and the conversion rate of carbon dioxide obtained when methane was produced through use of the reaction device 110 including the first reactor 210a and the second reactor 210b were simulated. In Example, hydrogen and carbon dioxide were supplied from the inlet 230a of the first reactor 210a, and carbon dioxide was supplied from the supply port 230b of the second reactor 210b. In addition, in Example, a simulation was performed for the case in which the ratio of hydrogen (moles) supplied from the inlet 230a to the total (moles) of carbon dioxide supplied from the inlet 230a and the supply port 230b was 4:1. A simulation was performed for the case in which the ratio of carbon dioxide supplied from the inlet 230a to carbon dioxide supplied from the supply port 230b was 30:70.

As Comparative Example, the temperature of a catalyst layer and the conversion rate of carbon dioxide obtained when methane was produced through use of one reactor having only the inlet 230a and the outlet 232c without a supply port were simulated. In Comparative Example, hydrogen and carbon dioxide were supplied from the inlet 230a. In addition, in Comparative Example, a simulation was performed for the case in which the ratio of hydrogen (moles) to carbon dioxide (moles) was 4:1.

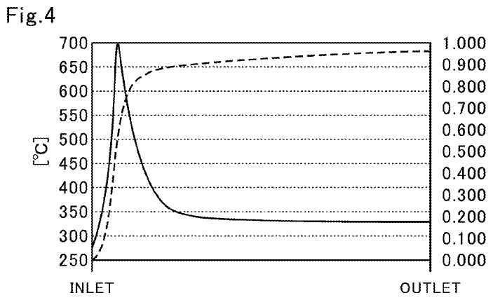

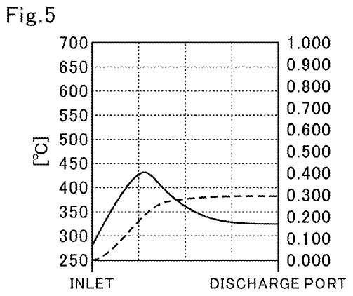

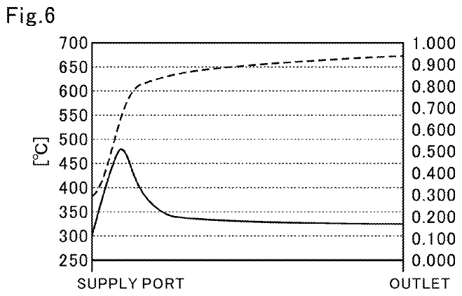

FIG. 4 is a graph for showing simulation results of Comparative Example. FIG. 5 is a graph for showing simulation results of the first reactor 210a in Example. FIG. 6 is a graph for showing simulation results of the second reactor 210b in Example. In FIG. 4 to FIG. 6, the left vertical axis indicates temperature [° C.], and the right vertical axis indicates the conversion rate of carbon dioxide. In addition, in FIG. 4 to FIG. 6, the solid line indicates the temperature of the catalyst layer, and the broken line indicates the conversion rate of carbon dioxide.

As shown in FIG. 4, in Comparative Example, it was confirmed that the temperature of the catalyst layer rose to about 700° C. Thus, in Comparative Example, it was found that the catalyst layer deteriorated due to heat.

Meanwhile, as shown in FIG. 5, in Example, it was confirmed that the temperature of the catalyst layer rose only to about 430° C. in the first reactor 210a. In addition, as shown in FIG. 6, in Example, it was confirmed that the temperature of the catalyst layer rose only to about 480° C. in the second reactor 210b. Thus, in Example, it was found that the deterioration of the catalyst layer due to heat was able to be suppressed.

In addition, as shown in FIG. 6, in Example, it was confirmed that the conversion rate of carbon dioxide in the second reactor 210b was comparable to that in Comparative Example. Thus, in Example, it was found that carbon dioxide was able to be efficiently converted into methane while the deterioration of the catalyst layer due to heat was suppressed.

The embodiments have been described above with reference to the attached drawings, but, needless to say, the present disclosure is not limited to the embodiments. It is apparent that those skilled in the art may arrive at various alternations and modifications within the appended claims, and those examples are construed as naturally falling within the technical scope of the present disclosure.

For example, in the above-mentioned first and second embodiments, there has been given, as an example, the case in which the hydrocarbon production apparatuses 100 and 300 each include the temperature sensors T1, T2, and T3, and the flow rate control units 182 and 382 each control the flow rate of carbon dioxide supplied by the carbon dioxide supply unit 130 to the first reactor 210a, the second reactor 210b, and the third reactor 210c, respectively, based on the detected values of the temperature sensors T1, T2, and T3. However, the hydrocarbon production apparatuses 100 and 300 may include no temperature sensors T1, T2, and T3. In this case, memories (not shown) of the hydrocarbon production apparatuses 100 and 300 each store information indicating a relationship between the supply amount of carbon dioxide to the first reactor 210a and the temperature of the catalyst layer of the first reactor 210a, information indicating a relationship between the supply amount of carbon dioxide to the second reactor 210b and the temperature of the catalyst layer of the second reactor 210b, and information indicating a relationship between the supply amount of carbon dioxide to the third reactor 210c and the temperature of the catalyst layer of the third reactor 210c, which are set in advance by simulation or the like. Then, the flow rate control units 182 and 382 each refer to the information indicating the relationship between the supply amount of carbon dioxide to the first reactor 210a and the temperature of the catalyst layer of the first reactor 210a, which is held in the memories, and adjust the opening degree of the flow rate adjustment valve 138a such that the catalyst layer of the first reactor 210a falls within the reaction temperature range. Similarly, the flow rate control units 182 and 382 each refer to the information indicating the relationship between the supply amount of carbon dioxide to the second reactor 210b and the temperature of the catalyst layer of the second reactor 210b, which is held in the memories, and adjust the opening degree of the flow rate adjustment valve 138b such that the catalyst layer of the second reactor 210b falls within the reaction temperature range. In addition, the flow rate control units 182 and 382 each refer to the information indicating the relationship between the supply amount of carbon dioxide to the third reactor 210c and the temperature of the catalyst layer of the third reactor 210c, which is held in the memories, and adjust the opening degree of the flow rate adjustment valve 138c such that the catalyst layer of the third reactor 210c falls within the reaction temperature range.

In addition, the flow rate control units 182 and 382 may each control the flow rate of carbon dioxide supplied by the carbon dioxide supply unit 130 based on the concentration of hydrocarbon contained in the generated gas discharged from the piping 240c. That is, the flow rate control units 182 and 382 may each adjust the opening degrees of the flow rate adjustment valves 138a, 138b, and 138c based on the concentration of hydrocarbon contained in the generated gas discharged from the piping 240c. With this configuration, the inside of the reaction device 110 can be set to an appropriate temperature.

In addition, the flow rate control units 182 and 382 may each control the temperature of the catalyst layer accommodated in the reaction device 110 by controlling the flow rate of carbon dioxide.

In addition, in the above-mentioned first and second embodiments, there has been given, as an example, the case in which the reaction device 110 of each of the hydrocarbon production apparatuses 100 and 300 has two supply ports 230b and 230c. However, the reaction device 110 may have one supply port or may have three or more supply ports. In addition, when the reaction device 110 has one supply port, the distance between the inlet and the supply port having a shortest distance from the inlet may be set to be shorter than the distance between the outlet and the supply port having a shortest distance from the outlet. In addition, when the reaction device 110 has three or more supply ports, the distance between the supply ports adjacent to each other may increase from the inlet 230a side toward the outlet 232c side. For example, the length of the reaction tube 214 may be increased toward the rear stage. With this configuration, the residence time of the gas can be increased from the inlet 230a toward the outlet 232c, thereby being capable of improving the conversion rate of carbon dioxide.

In addition, in the above-mentioned first and second embodiments, there has been given, as an example, the case in which the hydrocarbon production apparatuses 100 and 300 each include the coolers 150a, 150b, and 150c. However, it is sufficient that the hydrocarbon production apparatuses 100 and 300 each include at least one cooler 150 among the coolers 150a, 150b, and 150c. In addition, the hydrocarbon production apparatuses 100 and 300 may each include no cooler 150.

Similarly, in the above-mentioned first and second embodiments, there has been given, as an example, the case in which the hydrocarbon production apparatuses 100 and 300 each include the gas-liquid separators 160a, 160b, and 160c. However, it is sufficient that the hydrocarbon production apparatuses 100 and 300 each include at least one gas-liquid separator 160 among the gas-liquid separators 160a, 160b, and 160c. In addition, the hydrocarbon production apparatuses 100 and 300 may each include no gas-liquid separator 160.

In addition, in the above-mentioned first and second embodiments, there has been given, as an example, the case in which the reactor 210 is a multi-tube type and heat-exchange-type reactor. However, the reactor 210 may be an adiabatic reactor. In addition, in the above-mentioned modification example, there has been given, as an example, the case in which the reactor 420 is an adiabatic reactor. However, the reactor 420 may be a multi-tube type and heat-exchange-type reactor.

In addition, in the above-mentioned first and second embodiments, the gas supplied to the reaction device 110 may be changed from carbon dioxide to carbon monoxide. In the exothermic reaction by the catalyst, carbon monoxide has better reactivity with hydrogen than carbon dioxide, and hence the temperature of the reaction device 110 can be raised rapidly in the activation process. Thus, the activation time can be further reduced by changing the gas supplied to the reaction device 110 from carbon dioxide to carbon monoxide.

In addition, in the above-mentioned first and second embodiments, carbon monoxide may be supplied to the reaction device 110 in addition to hydrogen and carbon dioxide. As described above, in the exothermic reaction by the catalyst, carbon monoxide has better reactivity with hydrogen than carbon dioxide, and hence the temperature of the reaction device 110 can be raised rapidly in the activation process. Thus, in the activation process, with carbon monoxide being mainly suppled to the reaction device 110 and carbon dioxide being then suppled after the temperature of the reaction device 110 is raised, carbon dioxide can be stably and continuously reacted.

Further, gases with different combinations of carbon dioxide and carbon monoxide (only carbon dioxide, only carbon monoxide, or a mixed gas of carbon dioxide and carbon monoxide) may be supplied to the plurality of reactors 210 described above, respectively, in addition to hydrogen. In this case, a desired temperature rise rate can be obtained in accordance with the temperature rise state and the like of each of the reactors 210, and hence efficient operation of the reactors 210 is enabled.

When hydrogen and carbon monoxide are supplied to the reaction device 110, the synthesis reaction of hydrogen and carbon monoxide is performed in the reaction device 110 to produce hydrocarbon. The synthesis reaction between hydrogen and carbon monoxide is an exothermic reaction. For example, the synthesis reaction between hydrogen and carbon monoxide is a reaction represented by formulae (5) to (8) below.

3H₂+CO→CH₄+H₂O Formula (5)

4H₂+2CO→C₂H₄+2H₂O Formula (6)

6H₂+3CO→C₃H₆+3H₂O Formula (7)

mH₂+nCO→hydrocarbon+nH₂O Formula (8)

The present disclosure can contribute to, for example, Goal 7 “Ensure access to affordable, reliable, sustainable and modern energy for all” and Goal 13 “Take urgent action to combat climate change and its impacts” in Sustainable Development Goals (SDGs).

What is claimed is:

1. A hydrocarbon production apparatus, comprising:
a reaction device, which has an inlet, an outlet, and one or a plurality of supply ports formed between the inlet and the outlet, and in which a catalyst that promotes an exothermic reaction between hydrogen and carbon dioxide is accommodated;
a first hydrogen supply unit that supplies the hydrogen into the reaction device through the inlet of the reaction device; and
a carbon dioxide supply unit that supplies the carbon dioxide into the reaction device through the inlet and the one or the plurality of supply ports of the reaction device.

2. The hydrocarbon production apparatus according to claim 1, further comprising a flow rate control unit that controls a flow rate of the carbon dioxide supplied by the carbon dioxide supply unit.

3. The hydrocarbon production apparatus according to claim 2, wherein the flow rate control unit controls the flow rate of the carbon dioxide based on a temperature of the catalyst accommodated in the reaction device.

4. The hydrocarbon production apparatus according to claim 1, wherein a distance between the inlet and the supply port having a shortest distance from the inlet is shorter than a distance between the outlet and the supply port having a shortest distance from the outlet.

5. The hydrocarbon production apparatus according to claim 4,
wherein the reaction device has two or more supply ports,
wherein the distance between the inlet and the supply port having a shortest distance from the inlet is shorter than a distance between the supply ports adjacent to each other, and
wherein the distance between the outlet and the supply port having a shortest distance from the outlet is longer than a distance between the supply ports adjacent to each other.

6. The hydrocarbon production apparatus according to claim 5,
wherein the reaction device has three or more supply ports, and
wherein the distance between the supply ports adjacent to each other increases from the inlet side toward the outlet side.

7. The hydrocarbon production apparatus according to claim 1,
wherein the reaction device includes a plurality of reactors connected in series,
wherein the inlet is formed in the reactor at a foremost stage, and
wherein the outlet is formed in the reactor at a rearmost stage.

8. The hydrocarbon production apparatus according to claim 7, further comprising a cooler that cools a gas discharged from a discharge port of at least any one of the plurality of reactors.

9. The hydrocarbon production apparatus according to claim 1, further comprising a second hydrogen supply unit that supplies the hydrogen into the reaction device through the one or the plurality of supply ports of the reaction device.

10. A hydrocarbon production apparatus, comprising:
a reaction device, which has an inlet, an outlet, and one or a plurality of supply ports formed between the inlet and the outlet, and in which a catalyst that promotes an exothermic reaction between hydrogen and carbon monoxide is accommodated;
a first hydrogen supply unit that supplies the hydrogen into the reaction device through the inlet of the reaction device; and
a carbon monoxide supply unit that supplies the carbon monoxide into the reaction device through the inlet and the one or the plurality of supply ports of the reaction device.

11. A hydrocarbon production apparatus, comprising:
a reaction device, which has an inlet, an outlet, and one or a plurality of supply ports formed between the inlet and the outlet, and in which a catalyst that promotes an exothermic reaction among hydrogen, carbon monoxide, and carbon dioxide is accommodated;
a first hydrogen supply unit that supplies the hydrogen into the reaction device through the inlet of the reaction device;
a carbon monoxide supply unit that supplies the carbon monoxide into the reaction device through the inlet and the one or the plurality of supply ports of the reaction device; and
a carbon dioxide supply unit that supplies the carbon dioxide into the reaction device through the inlet and the one or the plurality of supply ports of the reaction device.
